SEQUENCE LISTING (2 sequences)

SEQ ID NO: 1 - protein, 213 aa, Homo sapiens
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

SEQ ID NO: 2 - DNA, 1314 bp, Homo sapiens
cagtgattctcgtgcctcagcctcctgagtagccgaaattacagacgtgtgccaccatgcttggctaattttttggatttttagtagagatggggtttcactatgttggccaggctagtcttgaactcctggcctgaagcaatccgcccacctcagcctcccaaagtgctgagattataggcacgagccactacacctggccacaaaattctttaaagaagccaatcccatcctccctcaagagccaaggggccacctcaccctcttgttacagcagatcctgcctcccacagtcaccctgctcccaagtgcaacctctgtctgaccctgcatggtgtgcggtgccctcctgcctcaggccgcgaagaaggatctaagggcttggcttgtttgaaagaaccacaccccgaaagtaacatctttggagaaagtgatacaagagcttctgcacccacctgatagaggaagtccaaagggtgtgcgcacacacaatggtgcctgaagaagagcctcaagaccgagagaaaggactctggtggttccagttgaaggtctggtccatggcagtcgtatccatcttgctcctcagtgtctgtttcactgtgagttctgtggtgcctcacaattttatgtatagcaaaactgtcaagaggctgtccaagttacgagagtatcaacagtatcatccaagcctgacctgcgtcatggaaggaaaggacatagaagattggagctgctgcccaaccccttggacttcatttcagtctagttgctactttatttctactgggatgcaatcttggactaagagtcaaaagaactgttctgtgatgggggctgatctggtggtgatcaacaccagggaagaacaggatttcatcattcagaatctgaaaagaaattcttcttattttctggggctgtcagatccagggggtcggcgacattggcaatgggttgaccagacaccatacaatgaaaatgtcacattctggcactcaggtgaacccaataaccttgatgagcgttgtgcgataataaatttccgttcttcagaagaatggggctggaatgacattcactgtcatgtacctcagaagtcaatttgcaagatgaagaagatctacatataaatgaaatattctccctggaaatgtgtttgggttggcatccaccgttgtagaaagctaaattgattttttaatttatgtgtaagttttgtacaaggaatgcccctaaaatgtttcagcaggctgtcacctattacacttatgatataatccattcacacattcatttattcatttattcatt

(19) United States
(12) Patent Application Publication
(10) Pub. No.: US 2015/0238632 A1
(43) Pub. Date: Aug. 27, 2015

(54) Compositions and Methods for Delivery of Antigens to Plasmacytoid Dendritic Cells

(71) Applicant: UNIVERSITY OF WASHINGTON THROUGH ITS CENTER FOR COMMERCIALIZATION, Seattle, WA (US)

(72) Inventors: Edward A. Clark, Seattle, WA (US); Craig Chappell, Seattle, WA (US); Natalia V. Giltiay, Seattle, WA (US); Kevin Draves, Seattle, WA (US)

(21) Appl. No.: 14/426,623

(22) PCT Filed: Sep. 18, 2013
(86) PCT No.: PCT/US2013/060318

§ 371 Date: Mar. 6, 2015

Related U.S. Application Data
(60) Provisional application No. 61/702,648, filed on Sep. 18, 2012.

(51) Int. Cl. A61K 47/48; A61K 39/00; C07K 16/28

(52) U.S. Cl. CPC A61K 47/48561; C07K 16/2851; A61K 39/0005; A61K 2039/6056

(57) ABSTRACT

The present invention provides compositions of blood dendritic cell antigen 2 (BDCA2) targeting molecules coupled to heterologous antigens, and their use in treating and/or limiting disease.

### CROSS REFERENCE

This application claims priority to U.S. Provisional Patent application Ser. No. 61/702,648 filed Sep. 18, 2012, incorporated by reference herein in its entirety.

### STATEMENT OF GOVERNMENT RIGHTS

This invention was made with government support under AI52203, DE16381, F32AI081455 and RR00166, awarded by the National Institutes of Health. The U.S. government has certain rights in the invention.

### BACKGROUND

Plasmacytoid dendritic cells (pDCs) were first identified as natural interferon (IFN)-producing cells (IPCs) due to their ability to secrete high quantities of type 1 IFNs following either TLR7 or TLR9 engagement by ssRNA and ssDNA viruses, respectively. Since production of type 1 IFNs during infection is critical for establishing an anti-viral state in the host, pDCs are a key component of innate immunity. Little is known about targeting antigen to plasmacytoid dendritic cells (pDCs), or how to modulate pDC-driven antigen-specific immune responses.

### SUMMARY OF THE INVENTION

In a first aspect, the present invention provides compositions comprising

(a) a blood dendritic cell antigen 2 (BDCA2) targeting molecule; and

(b) a heterologous antigen directly attached to the BDCA2 targeting molecule.

In one embodiment, the composition does not include an adjuvant. In another embodiment, the composition does comprise an adjuvant. In various other embodiments, the heterologous antigen is a polysaccharide, glycolipid antigen, or polypeptide antigen. In another embodiment, the heterologous antigen is an antigen that can cause an undesirable immune response, such as an allergen or an autoimmune antigen. In a further embodiment, the BDCA2 targeting molecule is a BDCA2-specific antibody or antibody fragment, including but not limited to an UW80.1 monoclonal anti-BDCA2, deposited with the American Type Culture Collection under Accession No. ______.

In a second aspect, the present invention provides isolated nucleic acid encoding the composition of embodiment of the first aspect of the invention, where the antigen is a polypeptide antigen, nucleic acid vectors comprising the isolated nucleic acid, and recombinant host cells comprising the nucleic acid vector.

In third aspect, the present invention provides monoclonal UW80.1 anti-BDCA2 antibodies deposited with the American Type Culture Collection under Accession No. ______, or a hybridoma expressing UW80.1 anti-BDCA2, deposited with the American Type Culture Collection under Accession No. ______.

In a fourth aspect, the present invention provides pharmaceutical compositions, comprising a composition of any aspect or embodiment of the invention and a pharmaceutically acceptable carrier.

In a fifth aspect, the invention provides methods for making antibodies, comprising culturing the recombinant host cells of the invention under conditions suitable for expression of nucleic-acid encoded antibody compositions or recombinant antibody or antibody fragment; and

isolating the antibody composition or recombinant antibody or antibody fragment from the cultured cells.

In a sixth aspect, the present invention provides methods for treating or limiting development of a disorder, comprising administering to an individual at risk of a disorder or having a disorder, an amount effective to treat or limit development of the disorder of the composition of any aspect or embodiment of the invention, or a pharmaceutically acceptable salt thereof. In one embodiment, the individual has or is at risk of developing an allergy to the heterologous antigen or an autoimmune disorder relating to the heterologous antigen, and wherein the method comprises administering the composition, or a pharmaceutically acceptable salt thereof, in the absence of adjuvant, to induce immune tolerance in the subject to the heterologous antigen.

In a seventh aspect, the invention provides methods for treating or limiting development of a disorder, comprising administering to an individual at risk of a disorder or having a disorder, an amount effective to treat or limit development of the disorder of a composition of any embodiment of the invention, or a pharmaceutically acceptable salt thereof, where the administering is done in the presence of adjuvant, to generate an immune response against the heterologous antigen.

### DESCRIPTION OF THE FIGURES

FIG. 1. Single cell suspensions were obtained from B6.BDCA2 Tg mice and pDCs enriched from these cells using magnetic beads to which anti-PDCA-1 mAbs were attached. The pDC-enriched cells were then cultured (1×10⁶ cells/ml) for 24 hrs with CpG-A (ODN 2216, 20 μg/ml) and various mAbs (2 μg/ml) as indicated. Supernatants were then isolated and IFNα production determined by ELISA.

FIG. 2. BDCA2 Tg mice were injected i.v. with 2 μg fluorescently-labeled BDCA2 mAb (UW80.1) and spleens were analyzed 15 min. later by flow cytometry. 80-90% of Siglec-H+ PDCA1+ pDCs bound BDCA2 mAb, while there was no significant binding of BDCA2 mAb by CD19+ B cells.

FIG. 3. Schematic of the method for delivering Ags via BDCA2 to pDCs in vivo.

FIG. 4. OTII OVA-specific CD4+ T cells were inoculated into B6.BDCA2 Tg mice and one day later mice were inoculated i.v. with 10 μg OVA-BDCA2 mAb UW80.1 alone or in the presence of the adjuvant CpG-B (50 μg) or with 10 μg OVA conjugated to a mouse IgG1 non-binding isotype control mAb (G28-1 anti-human CD37, OVA-Iso) with CpG-B. IgG and IgG2b Ab responses 10 days after immunization were determined by ELISA.

FIG. 5. (A) Schematic flow of study. Mice were inoculated with OTII OVA-specific CD4 T cells on day −1 and 24 hrs later were inoculated i.v. with 1 μg of OVA-anti-BDCA2 or OVA-Iso Flow cytometric analyses illustrated in (B) and summarized in (C-D) showing frequencies and numbers of OVA-specific CD4+ Ly5.1+ T cells and OVA-specific Foxp3+ CD4+ Ly5.1+ T cells (C) and frequencies of Annexin V+ CD4+ Ly5.1+ T cells (D).

FIG. 6. (A) Schematic flow of study. Mice were injected day −1 with OTII CD4+ T cells i.v. and on day 0 i.v. with 1 ug of OVA-isotype or OVA-BDCA2 mAb; 14 days later mice received a boost with a high dose intraperitoneally of OVA/precipitated in the Alum adjuvant, and 7 days later mice were analyzed for their frequencies and numbers of Ag-specific T cells. Flow cytometric analyses illustrated in (B) and summarized in (C-E). In some experiments illustrated in (E) groups of mice also were day −1 with OTII CD4+ T cells i.v. and on day 0 i.v. with 1 ug of OVA-isotype2 or OVA-Single H mAb.

FIG. 7. (A) Schematic flow of study. Groups of mice were injected day −1 with OTII CD4+ T cells i.v. and on day 0 intraperitoneally with 50 μg of OVA/precipitated in the Alum adjuvant (positive control) or i.v. with PBS (negative control), 1 ug of OVA-isotype mAb (negative control) or 1 ug of OVA-BDCA2 mAb; 14 days later mice were immunized with OVA/precipitated in the Alum adjuvant or with chicken gammaglobulin (CGG) precipitated in the Alum adjuvant and were bled 7, 14, 21 and 28 days thereafter. The data are summarized in (B) and (C).

FIG. 8. (A) Schematic flow of study. After inducing Ag-specific tolerance via OVA-anti-BDCA2, we injected mice twice with a rat anti-CD25 mAb PC61.5.3 to reduce T regs or with a rat isotype control mAb (HRPN). These mice were then boosted with OVA/Alum and either followed for Ab responses or sacrificed 7 days later and assessed for CD4 T cell levels. The data are summarized in (B-E).

FIG. 9. A) Schematic flow of study. Mice were injected day −1 with OTII CD4+ T cells i.v. and on day 0 i.v. with either PBS, 1 ug of OVA-isotype or OVA-BDCA2 mAb; alone or with the TLR-7 adjuvant R848 at 50 μg. 14 days later mice received a boost intraperitoneally of 50 μg OVA/precipitated in Alum, and then 14 days later mice were analyzed for Ag-specific serum Ab levels. The data are summarized in (B).

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides compositions comprising

(a) a blood dendritic cell antigen 2 (BDCA2) targeting molecule; and

(b) a heterologous antigen directly attached to the BDCA2 targeting molecule.

BDCA2 is also known as C-type lectin domain family 4 member C (CLEC4C) or CD303. Its amino acid sequence (SEQ ID NO:2) and nucleic acid sequence (SEQ ID NO:1) are provided in the attached sequence listing.

The compositions of the invention can be used, for example, in methods of the invention targeting antigen to BDCA2, a surface protein expressed on pDCs, to surprising stimulate immune tolerance or immunity against the heterologous antigen, depending on whether the methods involve the use of adjuvant.

The BDCA2 targeting molecule may be any molecule that directly binds to BDCA2 present on the surface of pDCs. In various non-limiting embodiments, the BDCA2 targeting molecule may be a polypeptide, nucleic acid, organic molecule, etc. In various non-limiting embodiments, the targeting molecule may comprise or consists of nucleic acid aptamers that bind to BDCA2 or peptide mimetics of BDCA2 antibodies. In a particular embodiment, the BDCA2 targeting molecule comprises a polypeptide; in one non-limiting embodiment, the polypeptide is an antibody or antibody fragment. As used herein, “antibody” includes reference to an immunoglobulin molecule immunologically reactive with human BDCA2 (preferably selective for BDCA2), and includes monoclonal antibodies. Various isotypes of antibodies exist, for example IgG1, IgG2, IgG3, IgG4, and other Ig, e.g., IgM, IgA, IgE isotypes. The term also includes genetically engineered forms such as chimeric antibodies (e.g., humanized murine antibodies) and heteroconjugate antibodies (e.g., bispecific antibodies), fully humanized antibodies, and human antibodies. As used throughout the application, the term “antibody” includes fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301. Various antigen binding domain-fusion proteins are also disclosed, e.g., in US patent application Nos. 2003/0118592 and 2003/0133939, and are encompassed within the term “antibody” as used in this application.

An antibody immunologically reactive with human BDCA2 can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

In one embodiment, the antibody comprises or consists of UW80.1 monoclonal anti-BDCA2 (described in the examples that follow), deposited with the American Type Culture Collection under Accession No. ______. As shown in the examples that follow, UW80.1 anti-BDCA2 partially inhibits CpG-A induced IFNα production, unlike a commercially available anti-BDCA2 mAb (Miltenyi, Inc.), which completely blocked IFNα production. As such, UW80.1 monoclonal anti-BDCA2 is particularly useful in the methods of the invention, particularly methods for generating an immune response against the heterologous antigen.

The compositions comprise a “heterologous” antigen, in that the antigen is not naturally present in the BDCA2 targeting molecule; thus, the compositions comprise a conjugate of the BDCA2 targeting molecule and the heterologous antigen. The BDCA2 targeting molecule and the heterologous antigen can be conjugated in any suitable manner, including covalently and non-covalently. Any linkage that irreversibly links the heterologous Ag to the BDCA2 targeting molecule can be used. The specific manner of conjugation most appropriate for a specific composition will depend on the components utilized, and can be determined by those of skill in the art. In some embodiments, the targeting molecule and the antigen can be conjugated via one or more linker moieties. In embodiments where both the targeting molecule and the antigen are polypeptides, the compositions can be recombinantly expressed as a chimera. Alternatively, any standard conjugation method can be used, including but not limited to engineered disulfide linkages, formalin/glutaradehyde crosslinking, streptavidin/biotin, etc.

The compositions may comprise any suitable antigen, since they can be used, for example, in methods to stimulate immune tolerance or to generate immunity against the antigen (depending on whether adjuvant is used). The heterologous antigen may be of any compound type, including but not limited to polypeptide, nucleic acid, lipid, polysaccharide, glycolipid, etc. In one embodiment the heterologous antigen comprise a polypeptide. In other embodiments, the heterologous antigen comprises a polysaccharide or a glycolipid antigen.

In one embodiment, particularly useful when used in methods to generate immunity against the antigen, the heterologous antigen comprises or consists of a pathogen-specific antigen; the antigen may be from any pathogen of interest in a given situation. In non-limiting embodiments, the pathogen-specific antigens include antigens from hepatitis (A, B, C, E, etc.) virus, human papillomavirus, herpes simplex viruses, cytomegalovirus, Epstein-Barr virus, influenza virus, parainfluenza virus, enterovirus, measles virus, mumps virus, polio virus, rabies virus, human immunodeficiency virus, respiratory syncytial virus, Rotavirus, rubella virus, varicella zoster virus, Ebola virus, cytomegalovirus, Marburg virus, norovirus, variola virus, any Flavivus including but not limited to West Nile virus, yellow fever virus, dengue virus, tick-borne encephalitis virus, and Japanese encephalitis virus; human immunodeficiency virus (HIV), Bacillus anthracis, Bordetalla pertusis, Chlamydia trachomatis, Clostridium tetani, Clastridium difficile, Corynebacterium diptheriae, Coxiella burnetii, Escherichia coli, Haemophilus influenza, Helicobacter pylori, Leishmania donovani, L. tropica and L. braziliensis, Mycobacterium tuberculosis, Mycobacterium leprae, Neisseria meningitis, Plasmodium falciparum, P. ovale, P. malariae and P. vivax, Pseudomonas aeruginosa, Salmonella typhi, Schistosoma hematobium, S. mansoni, Streptococcus pneumoniae (group A and B), Staphylococcus aureus, Toxoplasma gondii, Trypanosoma brucei, T. cruzi and Vibrio cholerae.

In a further embodiment, the heterologous antigen comprises or consists of other types of disease-related antigens against which it would be beneficial to generate an immune response against, including but not limited to antigens expressed in or on the surface of tumors/tumor cells (including but not limited to p53 (colorectal cancer), alphafetoprotein (germ cell tumors; hepatocellular carcinoma), carcinoembryonic antigen (bowel cancers), CA-125 (ovarian cancer), human epidermal growth factor receptor-2 (HER-2, breast cancer), MUC-1 (breast cancer), NY-ESO-1 (esophageal cancer, non-small-cell lung cancer), epithelial tumor antigen (breast cancer), tyrosinase (malignant melanoma), disialoganglioside (GD2, neuroblastoma), melanoma-associated antigen gene-1 (MAGE-1 (malignant melanoma)), and beta amyloid (for Alzheimer's and other amyloid-based diseases), etc.

In another embodiment, particularly useful when used in methods to promote immune tolerance to the antigen, the heterologous antigen comprises or consists of an allergen (i.e., any substance that can cause an allergy), including but not limited to allergens found in animal products (fur and dander; wool, dust mite excretion, etc.); drugs (penicillin, sulfonamides, salicylates, etc.); food (celery, corn, eggs/albumin, fruit, milk/lactose, seafood, sesame, legumes (beans, peas, peanuts, soybeans, etc); soy, tree nuts (pecans, almonds, etc.)); insect stings (bee sting venom, wasp sting venom, mosquito stings, etc.); mold spores such as from the fungus Alternaria alternata, and plant pollens (grass, weeds, ragweed, trees, etc.).

In another embodiment, particularly useful when used in methods to promote immune tolerance to the antigen, the heterologous antigen comprises or consists of a protein used for therapy, which is injected into patients deficient in that protein, who normally make antibodies to the therapeutic protein, including but not limited to recombinant human factor VIII (hemophilia), recombinant factor IX, lysosomal acid a glucosidase (Pompe's disease), α-galactosidase A, α-1-iduronidase or other enzymes used in enzyme-replacement therapy, immunoglobulin (Ig) or Ig domains, insulin, tumor necrosis factor-alpha to prevent the development of anti-drug-antibodies (ADA).

In a further embodiment, particularly useful when used in methods to promote immune tolerance to the antigen, the heterologous antigen comprises or consists of an autoimmune antigen, such as antigens involved in diabetes mellitus, multiple sclerosis, Crohn's disease, inflammatory bowel disease, rheumatoid arthritis, thyroiditis, vitiligo, and/or systemic lupus erythematosis. The heterologous antigen can be an antigen involved in allograft or graft rejection, such that the induced tolerance reduces the allograft rejection. In various non-limiting embodiments, the autoimmune antigen can include (relevant autoimmune disorder shown in parentheses), but is not limited to, interferon, transglutaminase, aromatic acid carboxylase, GAD, HAI, 17-hydroxylase, and 21 hydroxlase (Addison's disease); cardiolipin, pyruvate dehydrogenase, apoH, β2 glycoprotein I, phosphatidylserine, and annexin A5 (antiphospholipid syndrome); soluble liver antigen (autoimmune hepatitis); lactoferrin, carbonic anhydrase, and rheumatoid factor (autoimmune pancreatitis); gpIIb-IIIa or 1b-IX (autoimmune thrombocytopenic purpura); GQ1b (Bickerstaff's encephalitis); type XVII collagen (bullous pemphigoid); transglutaminase, gliadin, and endomysial (celiac disease); gangliosides, GM-1 gangliosides, GD1a gangliosides, GQ1b gangliosides (chronic inflammatory demyelinating polyneuropathy); BP-1 or BP-2 (cicatricial pemphigoid); epidermal transglutaminase (dermatitis herpetiformis); histidine tRNA, JO-1, or Mi-2 (dermatomyositis); glutamic acid decraboxylase or insulin (type 1 diabetes); sc170 or topoisomerase (diffuse cutaneous systemic sclerosis); histones (drug-induced lupus); type IV collagen (Goodpasture syndrome); gangliosides (Guillain-Barre' syndrome); enolase (Hashimoto's encephalopathy); thyroid peroxidase or thyroglobulin (Hashimoto's thyroiditis); complement component 3 (Henoch-Schonlein purpura); gangliosides (chronic inflammatory demyelinating polyneuropathy); rheumatoid factor (juvenile rheumatoid arthritis); synaptogagmin or muscarinic acetylcholine receptor M1 (Lambert-Eaton myasthenic syndrome); Ro (lupus erythematosus; Sjogren's syndrome); major peripheral myelin protein P0 (Ménière's disease); myeloperoxidase (microscopic polyangiitis); U1 RNP components (mixed connective tissue disease); KIR4.1 (multiple sclerosis); nicotinic acetycholine receptor MuSK protein (myaasthenia gravis); hypocretin or orexine (narcolepsy); aquaporin 4 (neuromyelitis optica); cyclic citrullinated peptide (palindromic rheumatism; others); Yo, Hu, Tr, or glutamate receptor (paraneoplastic cerebellar degeneration); desmoglein 3 (pemphigus vulgaris); interferon gamma, interleukin-1, or tumor necrosis factor-alpha (polymyositis); p62, sp100, or Ro (primary biliary cirrhosis); HLA-DR52a (primary sclerosing cholangitis); NR2A (Rasmussen's encephalitis); rheumatoid factor or mutated citrullinated vimentin (rheumatoid arthritis); myosin (rheumatic fever); 21 hydroxylase or 17 hydroxylase (Schmidt syndrome); topoisomerase I (scleroderma); Ro (Sjögren's syndrome); and C1q (urticarial vasculitis); or antigenic fragments of any of the above.

As discussed herein, the inventors have surprisingly discovered that the compositions of the invention can be used to stimulate immune tolerance to the heterologous antigen when administered in the absence of adjuvant, while promoting immunity against the heterologous antigen when administered in the presence of adjuvant. Thus, in one embodiment, the compositions of the invention do not include an adjuvant; this embodiment is particularly suited for use in methods to stimulate immune tolerance to the heterologous antigen.

In another embodiment, the compositions further comprise an adjuvant; this embodiment is particularly suited for use in methods to promote immunity against the heterologous antigen. Any suitable adjuvant can be used in this embodiment, including but not limited to aluminum salts (alum), squalene-in-water emulsions, forms of detoxified monophosphoryl lipid A (MPL) or other Toll-like receptor (TLR) family agonists such as Poly-IC, flagellin, imiquimods, CpGs oligodeoxynucleotides or saponins, Complete Freund's adjuvant (CFA), incomplete Freund's adjuvant, CAF01 trehalose dimycolate, mineral or paraffin oil+surfactant, and combinations thereof. Other suitable formulations can be found, for example, in Immunity. 2010 Oct. 29; 33(4):492-503 and Curr Opin Immunol. 2012 June; 24(3):310-5.

In another embodiment, the compositions of the invention can be modified to extend half-life, such as by attaching at least one molecule to the composition for extending serum half-life, including but not limited to a polyethlyene glycol (PEG) group, serum albumin, transferrin, transferrin receptor or the transferrin-binding portion thereof, or combinations thereof. As used herein, the word “attached” refers to a covalently or non-covalently conjugated substance. The conjugation may be by genetic engineering or by chemical means.

The compositions of the present invention may be stored in any suitable buffer that is safe and well tolerated for the proposed uses of the compositions.

In a second aspect, the present invention provides isolated nucleic acids encoding the compositions of the invention when the targeting molecule and the antigen are polypeptides. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals.

In third aspect, the present invention provides monoclonal UW80.1 anti-BDCA2 antibodies deposited with the American Type Culture Collection under Accession No. ______, or a hybridoma expressing UW80.1 anti-BDCA2, deposited with the American Type Culture Collection under Accession No. ______. The monoclonal antibodies produced by these hybridoma clones can be cultured/propagated/stored in any suitable medium, as will be understood by those of skill in the art.

In a fourth aspect, the present invention provides nucleic acid vectors comprising the isolated nucleic acid of the third aspect of the invention. “Recombinant expression vector” includes vectors that operatively link a nucleic acid coding region or gene to any promoter capable of effecting expression of the gene product. The promoter sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In a fifth aspect, the present invention provides recombinant host cells comprising the nucleic acid vector of the fourth aspect of the invention. The host cells can be either prokaryotic or eukaryotic. The cells can be transiently or stably transfected. Such transfection of expression vectors into prokaryotic and eukaryotic cells (including but not limited to Chinese hamster ovary (CHO) cells) can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.).

The recombinant host cells can be used, for example in methods for producing antibody (when the targeting molecule is an antibody, or for recombinant host cells expressing the recombinant BDCA2 antibodies or antibody fragments of the invention), comprising:

(a) culturing the recombinant host cell of the invention under conditions suitable for expression of the nucleic-acid encoded antibody composition; and

(b) isolating the antibody composition from the cultured cells.

Suitable conditions for expression of the nucleic-acid encoded antibody composition can be determined by those of skill in the art based on the teachings herein, the specific host cells and vectors used, and the general knowledge of those of skill in the art.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes disclosed herein. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes disclosed herein.

In a sixth aspect, the present invention provides pharmaceutical compositions, comprising:

(a) the composition of any embodiment or combination of embodiments disclosed herein; and

(b) a pharmaceutically acceptable carrier.

In this aspect, the compositions are combined with a pharmaceutically acceptable carrier. Suitable acids which are capable of forming such salts include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming such salts include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

The pharmaceutical composition may comprise in addition to the composition of the invention (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The pharmaceutical compositions of the invention may be made up in any suitable formulation, preferably in formulations suitable for administration by injection. Such pharmaceutical compositions can be used, for example, in methods of use as vaccines, prophylactics, or therapeutics.

The pharmaceutical compositions may contain any other components as deemed appropriate for a given use, such as additional therapeutics or vaccine components.

In a seventh aspect, the present invention provides methods for treating or limiting development of a disorder, comprising administering to an individual at risk of a disorder an amount effective to treat or limit development of the disorder of the composition or pharmaceutical composition, or a pharmaceutical salt thereof, of any embodiment or combination of embodiments of the present invention. The inventors have surprisingly discovered that the compositions of the invention can be used to stimulate immune tolerance to the heterologous antigen when administered in the absence of adjuvant, while promoting immunity against the heterologous antigen when administered in the presence of adjuvant. The compositions of the invention induce reduction in antigen (Ag)-specific effector T cells while at the same time activating Ag-specific regulatory T cells (T reg cells), which mediate the tolerance. When used in combination with one or more adjuvants that activate/stimulate plasmacytoid dendritic cells, the compositions of the invention induce expansion of effector/helper CD4 T cells which provide necessary co-stimulation to B cells for induction of antibody responses.

In one embodiment, the individual has or is at risk of developing an allergy to the heterologous antigen or an autoimmune disorder relating to the heterologous antigen, and wherein the method comprises administering an appropriate composition of the invention, or a pharmaceutically acceptable salt thereof, in the absence of adjuvant, to induce immune tolerance in the subject to the heterologous antigen. In this embodiment, the methods result in reduction of an immune response. As used herein, reduction of an immune response refers to a lessening of one or more effects produced by the immune system (cellular or humoral) in reaction to the heterologous antigen leading to a prophylactic or therapeutic effect. Such reduction includes both reduction of an actual immune response or a reduction in an immune response that would otherwise have been expected in the absence of the reducing treatment. Reduction can include, but does not require, lessening of the immune response to an undetectable level.

In one such embodiment, the individual has an autoimmune disorder and the immune tolerance may comprise a reduced auto-immune response in the individual with an autoimmune disorder. Any level of reduction in autoimmune response compared to in the absence of treatment with the compositions of the invention is beneficial. In various embodiments, the reduction includes e.g., at least a 5% reduction in autoantibody titer, and preferably at least a 10%, 25%, 50%, or more reduction in autoantibody titer. In various embodiments, the heterologous antigen is an autoimmune antigen can include (relevant autoimmune disorder shown in parentheses), but is not limited to, interferon, transglutaminase, aromatic acid carboxylase, GAD, HAI, 17-hydroxylase, and 21 hydroxlase (Addison's disease); cardiolipin, pyruvate dehydrogenase, apoH, P2 glycoprotein I, phosphatidylserine, and annexin A5 (antiphospholipid syndrome); soluble liver antigen (autoimmune hepatitis); lactoferrin, carbonic anhydrase, and rheumatoid factor (autoimmune pancreatitis); gpIIb-IIIa or 1b-IX (autoimmune thrombocytopenic purpura); GQ1b (Bickerstaff's encephalitis); type XVII collagen (bullous pemphigoid); transglutaminase, gliadin, and endomysial (celiac disease); gangliosides, GM-1 gangliosides, GD1a gangliosides, GQ1b gangliosides (chronic inflammatory demyelinating polyneuropathy); BP-1 or BP-2 (cicatricial pemphigoid); epidermal transglutaminase (dermatitis herpetiformis); histidine tRNA, JO-1, or Mi-2 (dermatomyositis); glutamic acid decraboxylase or insulin (type 1 diabetes); sc170 or topoisomerase (diffuse cutaneous systemic sclerosis); histones (drug-induced lupus); type IV collagen (Goodpasture syndrome); gangliosides (Guillain-Barre' syndrome); enolase (Hashimoto's encephalopathy); thyroid peroxidase or thyroglobulin (Hashimoto's thyroiditis); complement component 3 (Henoch-Schonlein purpura); gangliosides (chronic inflammatory demyelinating polyneuropathy); rheumatoid factor (juvenile rheumatoid arthritis); synaptogagmin or muscarinic acetylcholine receptor M1 (Lambert-Eaton myasthenic syndrome); Ro (lupus erythematosus; Sjogren's syndrome); major peripheral myelin protein P0 (Ménière's disease); myeloperoxidase (microscopic polyangiitis); U1 RNP components (mixed connective tissue disease); KIR4.1 (multiple sclerosis); nicotinic acetycholine receptor MuSK protein (myaasthenia gravis); hypocretin or orexine (narcolepsy); aquaporin 4 (neuromyelitis optica); cyclic citrullinated peptide (palindromic rheumatism; others); Yo, Hu, Tr, or glutamate receptor (paraneoplastic cerebellar degeneration); desmoglein 3 (pemphigus vulgaris); interferon gamma, interleukin-1, or tumor necrosis factor-alpha (polymyositis); p62, sp100, or Ro (primary biliary cirrhosis); HLA-DR52a (primary sclerosing cholangitis); NR2A (Rasmussen's encephalitis); rheumatoid factor or mutated citrullinated vimentin (rheumatoid arthritis); myosin (rheumatic fever); 21 hydroxylase or 17 hydroxylase (Schmidt syndrome); topoisomerase I (scleroderma); Ro (Sjögren's syndrome); and C1q (urticarial vasculitis); or antigenic fragments of any of the above.

In another embodiment, the individual has an allergy and the immune tolerance may comprise a reduced allergic response in the individual. In this embodiment, the heterologous antigen may comprise any allergen, including but not limited to allergens found in animal products (fur and dander; wool, dust mite excretion, etc.); drugs (penicillin, sulfonamides, salicylates, etc.); food (celery, corn, eggs/albumin, fruit, milk/lactose, seafood, sesame, legumes (beans, peas, peanuts, soybeans, etc); soy, tree nuts (pecans, almonds, etc.)); insect stings (bee sting venom, wasp sting venom, mosquito stings, etc.); mold spores such as from the fungus Alternaria alternata, and plant pollens (grass, weeds, ragweed, trees, etc.).

In a further embodiment, the methods are used to promoting immunity against the heterologous antigen by stimulating an immune response. In this embodiment, the methods comprise administering an appropriate composition of the invention to an individual at risk of a disorder or having a disorder in the presence of adjuvant, wherein the method results in an enhancement of the immune response against the heterologous antigen. As used herein, enhancement of an immune response refers to an increase of one or more effects produced by the immune system in reaction to an antigen. Such enhancement includes enhancement of an actual immune response (prophylactic or therapeutic) or an enhancement in an immune response that would otherwise not have been expected in the absence of the enhancing treatment. As used herein, generation of an immune response refers to the creation of one or more effects produced by the immune system (to a detectable level) in reaction to an antigen. Such generation includes creation of an immune response that would otherwise not have been expected in the absence of the generating treatment.

In this embodiment, any suitable adjuvant can be used in this embodiment, including but not limited to aluminum salts (alum), squalene-in-water emulsions, forms of detoxified monophosphoryl lipid A (MPL) or other Toll-like receptor (TLR) family agonists such as Poly-IC, flagellin, imiquimods, CpGs oligodeoxynucleotides or saponins, complete Freund's adjuvant (CFA), incomplete Freund's adjuvant, CAF01 trehalose dimycolate, mineral or paraffin oil+surfactant, and combinations thereof. Other suitable formulations can be found, for example, in Immunity. 2010 Oct. 29; 33(4):492-503 and Curr Opin Immunol. 2012 June; 24(3):310-5. Similarly, the methods can be used to generate an immune response against any suitable antigen. In one embodiment, the compositions are used prophylactically as vaccines to limit infectious disease/severity of infectious disease, such as in individuals that have not been exposed to an infectious agent but are at risk of such exposure. In other embodiments, the compositions can be used therapeutically to treat people exposed to or chronically infected with a pathogen. As will be understood by those of skill in the art, the specific antigen/composition to be used will depend on the specific disorder to be treated or limited. Exemplary antigens for use in the methods of this embodiment include, but are not limited to pathogen-specific antigens including antigens from hepatitis (A, B, C, E, etc.) virus, human papillomavirus, herpes simplex viruses, cytomegalovirus, Epstein-Barr virus, influenza virus, parainfluenza virus, enterovirus, measles virus, mumps virus, polio virus, rabies virus, human immunodeficiency virus, respiratory syncytial virus, Rotavirus, rubella virus, varicella zoster virus, Ebola virus, cytomegalovirus, Marburg virus, norovirus, variola virus, any Flavivus including but not limited to West Nile virus, yellow fever virus, dengue virus, tick-borne encephalitis virus, and Japanese encephalitis virus; human immunodeficiency virus (HIV), Bacillus anthracis, Bordetalla pertusis, Chlamydia trachomatis, Clostridium tetani, Clastridium difficile, Corynebacterium diptheriae, Coxiella burnetii, Escherichia coli, Haemophilus influenza, Helicobacter pylori, Leishmania donovani, L. tropica and L. braziliensis, Mycobacterium tuberculosis, Mycobacterium leprae, Neisseria meningitis, Plasmodium falciparum, P. ovale, P. malariae and P. vivax, Pseudomonas aeruginosa, Salmonella typhi, Schistosoma hematobium, S. mansoni, Streptococcus pneumoniae (group A and B), Staphylococcus aureus, Toxoplasma gondii, Trypanosoma brucei, T. cruzi and Vibrio cholerae. In a further embodiment, the heterologous antigen comprises or consists of other types of disease-related antigens against which it would be beneficial to generate an immune response against, including but not limited to antigens expressed in or on the surface of tumors/tumor cells (including but not limited to p53 (colorectal cancer), alphafetoprotein (germ cell tumors; hepatocellular carcinoma), carcinoembryonic antigen (bowel cancers), CA-125 (ovarian cancer), human epidermal growth factor receptor-2 (HER-2, breast cancer), MUC-1 (breast cancer), NY-ESO-1 (esophageal cancer, non-small-cell lung cancer), epithelial tumor antigen (breast cancer), tyrosinase (malignant melanoma), Disialoganglioside (GD2, neuroblastoma), melanoma-associated antigen gene-1 (MAGE-1 (malignant melanoma)), and beta amyloid (for Alzheimer's and other amyloid-based diseases), etc. In another embodiment, particularly useful when used in methods to promote immune tolerance to the antigen, the heterologous antigen comprises or consists of a protein used for therapy, which is injected into patients deficient in that protein, who normally make antibodies to the therapeutic protein, including but not limited to recombinant human factor VIII (hemophilia), recombinant factor IX, lysosomal acid a glucosidase (Pompe's disease), α-galactosidase A, α-1-iduronidase or other enzymes used in enzyme-replacement therapy, immunoglobulin (Ig) or Ig domains, insulin, tumor necrosis factor-alpha to prevent the development of anti-drug-antibodies (ADA).

As used herein, “treat” or “treating” means accomplishing one or more of the following in an individual that already has a disorder or has already been exposed to a disorder-causing substance/pathogen: (a) reducing the severity of the disorder; (b) limiting or preventing development of symptoms characteristic of the disorder(s) being treated (ex: immune deficiencies in cancer patients or other patients) undergoing chemotherapy and/or radiation therapy); (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “limiting” or “limiting development of” means accomplishing one or more of the following in an individual that does not have the disorder to be limited: (a) preventing the disorder; (b) reducing the severity of the disorder; and (c) limiting or preventing development of symptoms characteristic of the disorder.

As used herein, an “amount effective” refers to an amount of the composition that is effective for treating and/or limiting the relevant disorder.

Suitable acids which are capable of forming pharmaceutically acceptable salts include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming pharmaceutically acceptable salts include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

For all methods disclosed herein, the compositions are typically formulated as a pharmaceutical composition for administration, such as those disclosed above, and can be administered via any suitable route, including orally, parentally, by inhalation spray, rectally, or topically in dosage unit formulations containing conventional pharmaceutically acceptable carriers, adjuvants, and vehicles. Preferably, the compositions are administered parenterally. The term parenteral as used herein includes, subcutaneous, intravenous, intra-arterial, intramuscular, intrasternal, intratendinous, intraspinal, intracranial, intrathoracic, infusion techniques or intraperitoneally. Dosage regimens can be adjusted to provide the optimum desired response (e.g., a therapeutic or prophylactic response). A suitable dosage range may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. The compositions can be delivered in a single bolus, or may be administered more than once (e.g., 2, 3, 4, 5, or more times) as determined by an attending physician. When adjuvant is used dosage regimens can be adjusted to lower the amount of targeted antigen used to provide the optimum desired response.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number, respectively. Additionally, the words “herein,” “above,” and “below” and words of similar import, when used in this application, shall refer to this application as a whole and not to any particular portions of this application. As used herein, the singular forms “a”, “an” and “the” include plural referents unless the context clearly dictates otherwise. “And” as used herein is interchangeably used with “or” unless expressly stated otherwise.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments.

All of the references cited herein are incorporated by reference. Aspects of the disclosure can be modified, if necessary, to employ the compositions, functions and concepts of the above references and application to provide yet further embodiments of the disclosure. These and other changes can be made to the disclosure in light of the detailed description.

The following description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure.

### EXAMPLES

### Introduction

Blood dendritic cell antigen-2 (BDCA2) is a type I/C-type lectin receptor (CLR) whose expression in humans is restricted to plasmacytoid dendritic cells (pDCs). Upon cross-linking with anti-BDCA2 mAb, BDCA2 is rapidly internalized to structures that resemble endosomes. BDCA2 cross-linking induces rapid Ca++ influx and phosphorylation of multiple signaling molecules including Syk, Vav1, PLCγ2, and Erk1/2 in an FccRIγ dependent manner. Although the precise signaling pathways initiated via BDCA2 cross-linking are not yet elucidated, they result in inhibition of IFN-α production induced by a variety of stimuli.

We investigated the utility of this receptor as a potential target for Ag delivery to pDCs. We utilized mice expressing a human BDCA2 construct (B6.BDCA2 transgenic mice) in pDCs together with a novel anti-BDCA2 mAb as a model system of Ag delivery to pDCs in vivo. In summary, these studies have demonstrated that:
B6.BDCA2 Tg mice display pDC-specific expression of BDCA2 and can be used as a model for Ag-delivery to pDCs;
Ag delivery to BDCA2⁺ pDCs without adjuvant does not induce Ag-specific T cell proliferation or Ab responses;
Targeting of Ag to BDCA2 on pDCs leads to reduction of Ag-specific T cells, but promotes an increase in the percentage of Ag-specific Foxp3⁺ T cells;
Prior delivery of Ag to BDCA2 on pDCs alters secondary T-cell responses and leads to significant increase in the percentage of Foxp3⁺ T cells after Ag-boost;
Ag delivery to BDCA2 on pDCs leads to inhibition of Ag-specific Ab responses following Ag re-challenge;
Depletion of CD25⁺ Foxp3⁺ T cells using anti-CD25 Ab restores Ag-specific Ab responses following Ag re-challenge; and
Treatment with TLR7 ligand at the time of Ag delivery “breaks” BDCA2-driven immune tolerance and restores Ag-specific Ab responses following Ag re-challenge.

Thus, these studies show that BDCA2-directed targeting of Ags to pDCs in the absence of adjuvant is a useful approach for promoting immune tolerance, such as in the treatment of autoimmune diseases, which involve activation of pathogenic Ag-specific T-cells and Ab production, and for promoting reduced Ab responses after re-exposure to Ags, such as in the treatment of allergic diseases, allergens, or in patients requirement protein replacement therapies, therapeutic proteins like factor VIII or glucosidase.

### Materials and Methods

### Animals

Mice expressing a BAC (#RP11-277J24) containing the BDCA2 gene including the endogenous promoter (RP11-277J24) were generated as in Kaplan et al. (Immunity, 23:611-620, 2005). These mice were crossed onto the H-2^{b} C57BL/6 strain (B6 background) for 8 generations to establish the BDCA2.B6 transgenic (Tg) line used in these studies. Transmission of the transgene was determined by polymerase chain reaction (PCR) using genomic DNA obtained from tail snips. C57BL/6 (B6) and B6.Ly5.1⁺ OT-II Tg mice with a T cell receptor (TCR) specific for MHC class II and a peptide derived from ovalbumin (OVA) were bred and maintained in our laboratory.

### Immunizations, Injections, and Adoptive Transfers

All intravenous injections were administered via the tail or retro-oribital veins in a 200 μl volume. Formulations of alum plus antigen were prepared according to manufacturer's instructions (Pierce, Inc.) and administered intraperitoneally in 100-200 μl volumes. When included, Toll-like receptor (TLR) agonists R848 (50 μg) or CpG-B (50 μg) (both from Invivogen) were admixed with the Ag and administered as a single i.v. injection. For adoptive transfers, splenocytes from Ly5.1⁺ OT-II TCR transgenic mice containing 1.5×10⁶ CD4⁺ Vα2⁺ T cells were injected i.v. into BDCA2.B6 recipients 1 day prior to immunization. All studies were pre-approved by the University of Washington's Institute for Animal Care and Use Committee.

### Antibodies, Reagents and Flow Cytometry.

Anti-BDCA2 clones UW80.1 and UW80.2 (both mouse IgG1) and mouse IgG1 clone G28-1 (negative control specific for human CD37) were generated and prepared from hybridomas established in our laboratory, using standard techniques. Briefly, we established NIH3T3 cell transfectants stably expressing BDCA2, and used these to generate and screen mouse mAbs against BDCA2. Two clones of the IgG1 isotype (UW80.1 and UW80.2) were identified that bound BDCA2⁺ NIH3T3 cell transfectants, but not empty vector controls. Analysis of both human and macaque PBMCs showed that UW80.1 bound to CD123⁺ pDCs, but not CD14⁺ monocytes (data not shown).

1-2×10⁶ mouse splenocytes prepared by mechanical disruption of spleen tissue or mouse peripheral blood mononuclear cells (PBMCs) from heparinized blood were incubated for 30 min on ice in FACS buffer (phosphate buffered saline (PBS) containing 2% fetal bovine serum) and varying combinations of biotin- or fluorochrome-conjugated mAbs against Siglec-H, PDCA-1, B220, CD11c, CD8, CD4, CD3, CD19, IgD, NK1.1, Vα2 TCR, Foxp3, CD25, CD44 (eBioscience), CD62L (BD Biosciences). Detection of BDCA2 was performed using Alexa Fluor 647-conjugated UW80.1 mAb (eBioscience AlexaFluor647 conjugation kit). Antibody-labeled cells were washed 3× with FACS buffer followed by detection of biotinylated mAbs using streptavidin-PerCP-Cy5.5 (eBioscience) or streptavidin-FITC (both from BD Biosciences) for 20 min on ice. Following washes, cells were re-suspended in PBS containing 1% PFA, and kept at 4 C in the dark until analysis.). For Foxp3 detection, the mouse Foxp3 staining kit (eBioscience) was used according to manufacturer's instructions. Apoptotic and dead cells were discriminated using AnnexinV (eBioscience) and APC-Cy7 live/dead discrimination reagent (Invitrogen, Inc.). Data was acquired using an LSR II or FACScan flow cytometer (BD Biosciences) and analyzed using FlowJo software (TreeStar, Inc.).

mAb-OVA Conjugate Preparation

OVA was conjugated to mAbs via thioether linkages as described (Handbook of Experimental Immunology, vol. 1, p 31. 6-31.7). Unconjugated OVA was removed from mAb-OVA conjugates using 100 kDa cut-off spin columns (Millipore, Inc.). Retained mAb-OVA conjugates were then sterile filtered (0.2 μM) and stored at −20 C until use. ELISA assays to detect mouse IgG1 and OVA (performed as above) were used to determine the final concentration of mAb and confirm mAb-Ag conjugation.

Purification of pDCs and In Vitro Stimulation

Single cell suspensions from spleens obtained from BDCA2.Tg mice were obtained using anit-mPDCA1 Micro bead isolation kit according to the manufacturer's instructions (Miltenyi Biotec Inc.). via treatment with Liberase RI (Roche), but otherwise according to the manufacturer's instructions. In some cases, spleens were mechanically disrupted in order to preserve the expression of Siglec-H on mouse pDCs. Red blood cells (RBCs) were lysed by incubating single cell suspensions in RBC lysis buffer containing 0.1M NH₄Cl, 0.015M NaCl, pH 7.4 for 2 min. Cells were washed twice in RPMI-1640 with 10% FCS and resuspended in PBS (phosphate-buffered saline) pH 7.2, 0.5% BSA, and 2 mM EDTA. Enriched pDCs were set up under sterile conditions in 24-well tissue culture plates at 1×10⁶/ml in RPMI-1640 with 50 μM EDTA and 10% FCS with either medium only, isotype control (G28-1, anti-human CD37), UW80.1 (anti-BDCA2), or a commercially available mAb (Miltenyi, anti-BDCA2) at either 2 μg/ml plus or minus 20 μg/ml CpG-A (ODN 2216) for 18 h in a 37° C., 5% CO2 humidified tissue culture incubator. The following day 800 μl of supernatants were removed from each well and assayed for IFNα production.

### ELISAs

Measurements of OVA and CGG-specific IgM or IgG Abs were performed as described using a modification of an ELISA (Goins, C. L., C. P. Chappell, R. Shashidharamurthy, P. Selvaraj, and J. Jacob. 2010 Immune complex-mediated enhancement of secondary antibody responses. J. Immunol. 184:6293-6298). High-capacity binding 96-well microplates were coated with a solution of 20 μg/ml of OVA or 20 μg/ml CGG (Sigma-Aldrich, Inc.) in PBS overnight at 4° C. Plates were blocked for 1 h at 37° C. using a solution of PBS containing 4% nonfat dry milk (NFDM) (Bio-Rad, Hercules, Calif.). Serum samples diluted in PBS-T and 0.1% NFDM were applied and allowed to react at room temperature for 1.5 h. Anti-mouse Abs with specificity for IgG, IgG1, or IgM isotypes coupled to HRP (Southern Biotech, Inc.) were applied and allowed to incubate for 1.5 h at room temperature. Absolute values were calculated from standard curves generated with known quantities of mouse IgG, IgG1, or IgM (Southern Biotech, Inc) captured by anti-mouse Ig(H+L) (Southern Biotech, Inc.). Detection of cytokines was performed using IFN-α detection kits according to the manufacturer's instructions (R&D Systems). For confirmation of OVA-mAb conjugation, mAb conjugates were captured as above with anti-mouse Ig(H+L) and detected with anti-OVA-biotin (Sigma-Aldrich, Inc.) followed by streptavidin-HRP (R&D Systems, Inc.). All plates were developed with tetramethylbenzidine substrate (Sigma-Aldrich, Inc.) and reactions were stopped with addition of equal volume 2N H₂SO₄. OD₄₅₀ values were obtained using a BioRad Model 550 microplate reader.

### Depletion of CD25⁺ T Cells In Vivo

Mice received two i.p. injections consisting of 500 μg rat anti-CD25 (clone PC61.5.3) or IgG1 isotype control Ab (HRPN) (BioXCell) at day 3 and day 1 prior to OVA/Alum challenge. Depletion of CD4+ Tregs was further confirmed by flow analysis by staining with anti-CD4, anti-Ly5.1 and anti-Foxp3 mAb that recognizes an epitope separate from that of clone PC61.5.3.

### Results

To assess the expression of BDCA2 in B6.BDCA2 transgenic (Tg) mice, spleen cells were isolated from C57BL/6 (B6) control mice (wildtype WT) or B6.BDCA-2 Tg mice, and using multicolor flow cytometry, the expression of BDCA2 was examined on various cell subsets. Staining for PDCA-1, a marker found on pDCs and BDCA2 revealed that BDCA2 is not expressed on the majority of B6 mouse spleen cells but is expressed on a population of PDCA-1+ cells from B6.BDCA2 Tg mice. These BDCA2+ cells, unlike CD11c^{hi} myeloid dendritic cells, express low levels of CD11c, and are Siglec-H+ and B220+, demonstrating they are pDCs. CD19+ B cells, CD4+ and CD8+ T cells, NK1.1+ NK cells and CD11c^{hi} cells did not express BDCA2, but SiglecH+PDCA1+ pDCs did. Thus, in B6.BDCA2 Tg mice, BDCA2 is only expressed on pDCs. No other cell type expressed BDCA2.

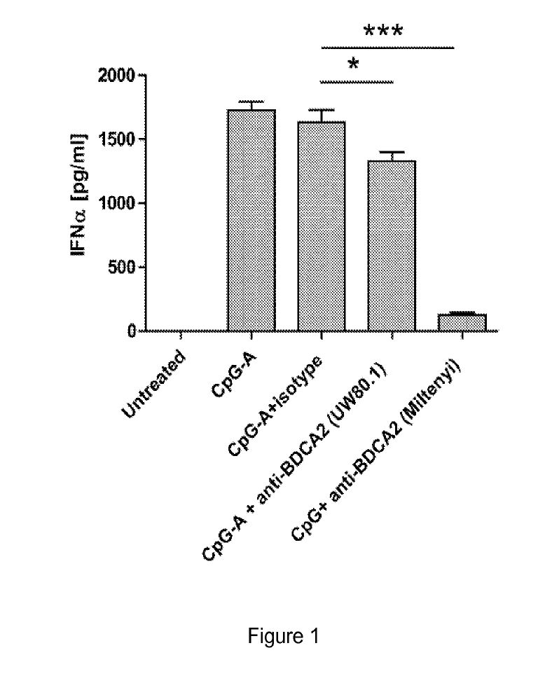

Single cell suspensions were obtained from B6.BDCA2 Tg mice and pDCs enriched from these cells using magnetic beads to which anti-PDCA-1 mAbs were attached. The pDC-enriched cells were then cultured (1×10⁶ cells/ml) for 24 hrs with CpG-A (ODN 2216, 20 μg/ml) and various mAbs (2 μg/ml) as indicated. Supernatants were then isolated and IFNα production determined by ELISA. Data is shown in FIG. 1. Compared to the isotype control UW80.1 anti-BDCA2 could partially inhibit CpG-A induced IFNα production, unlike a commercially available anti-BDCA2 mAb (Miltenyi, Inc.), which completely blocked IFNα production. Thus, the BDCA2+ mouse pDCs can be inhibited by anti-BDCA2 mAb, but the anti-BDCA2 mAbs differ in their properties Anti-BDCA2 mAb inhibited IFNα production by CpG-A stimulated pDCs, but not IL-12p40 production (data not shown), demonstrating that not all cytokines are affected by anti-BDCA2.

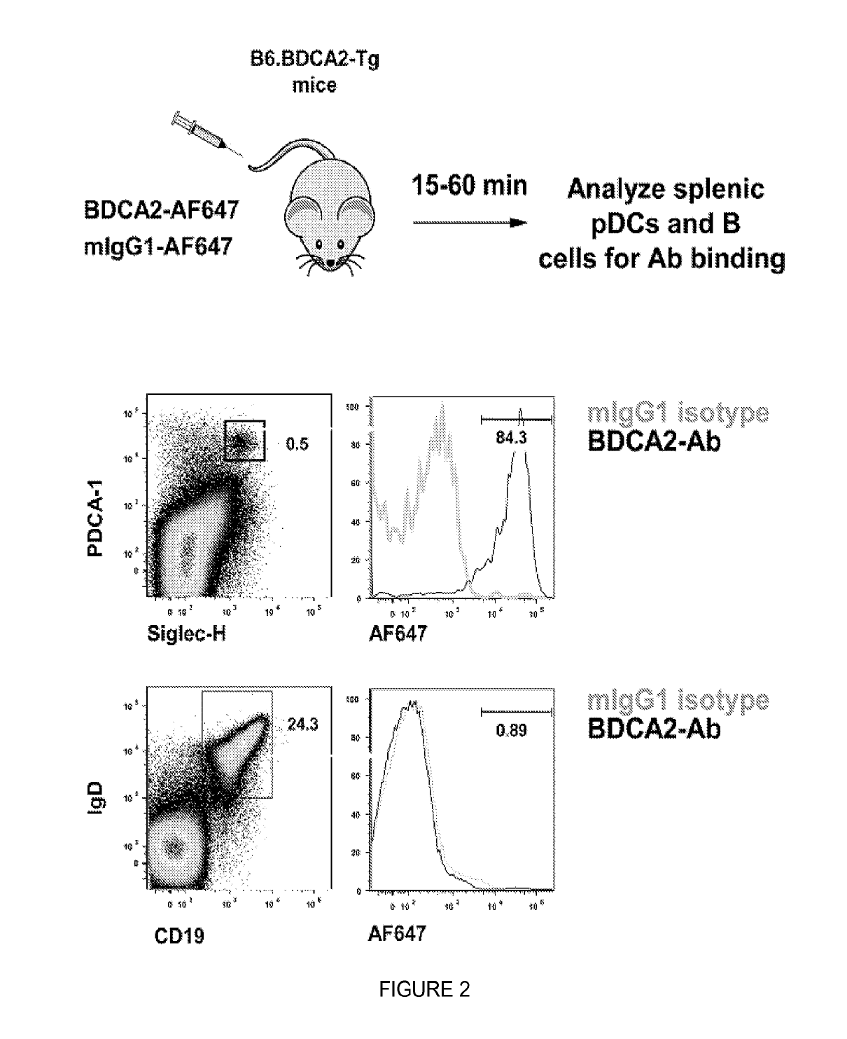

Intravenous injection of fluorescently-labeled BDCA2 mAb (UW80.1) labeled 80-90% of Siglec-H+ PDCA1+ pDCs (FIG. 2), while there was no significant binding of BDCA2 mAb by CD19+ B cells. Selective binding could be detected 15 minutes after administration; similar results were obtained when using UW80.2 anti-BDCA2 mAb. These data show that pDCs are selectively bound in vivo using anti-BDCA2 monoclonal Abs but not B cells, i.e., the targeting is restricted to pDCs.

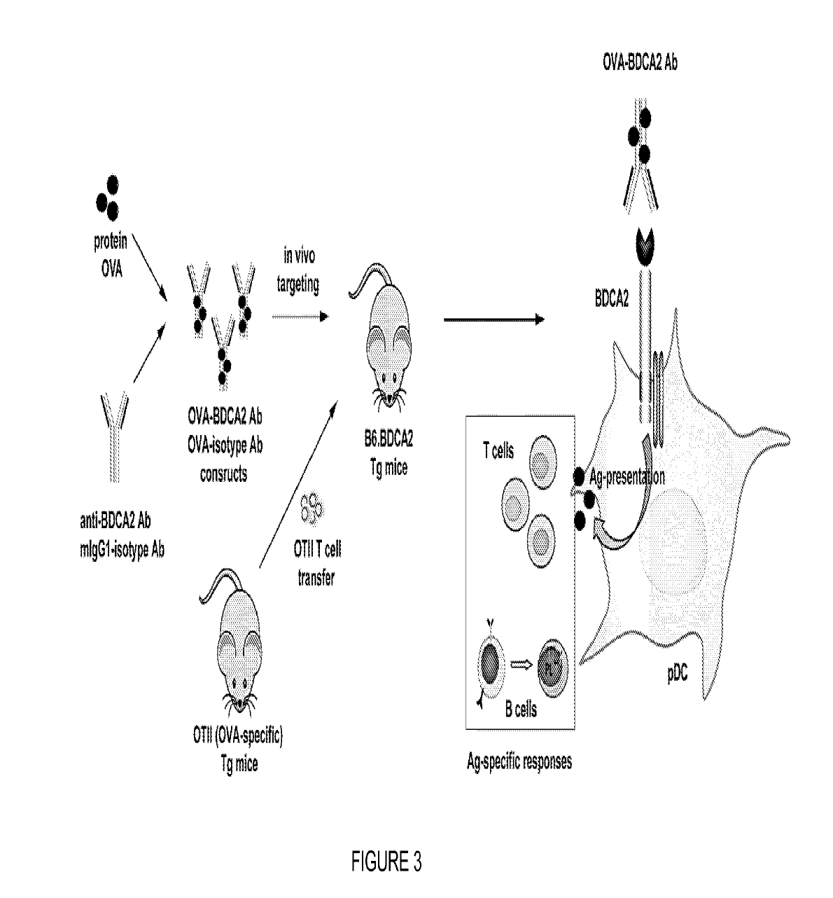

FIG. 3 shows a schematic of the method for delivering Ags via BDCA2 to pDCs in vivo. The protein ovalbumin (OVA) is coupled to anti-BDCA2 mAb, or an appropriate isotype control mouse IgG1 mAb and constructs are injected into BDCA2 Tg mice, OVA is delivered to the pDCs and OVA is subsequently presented to the T cells. OTII Tg mice are used as donors of congenic Ly5.1+ OVA-specific CD4+ T cells, which allowed us to study the OVA-specific CD4+ T cell responses.

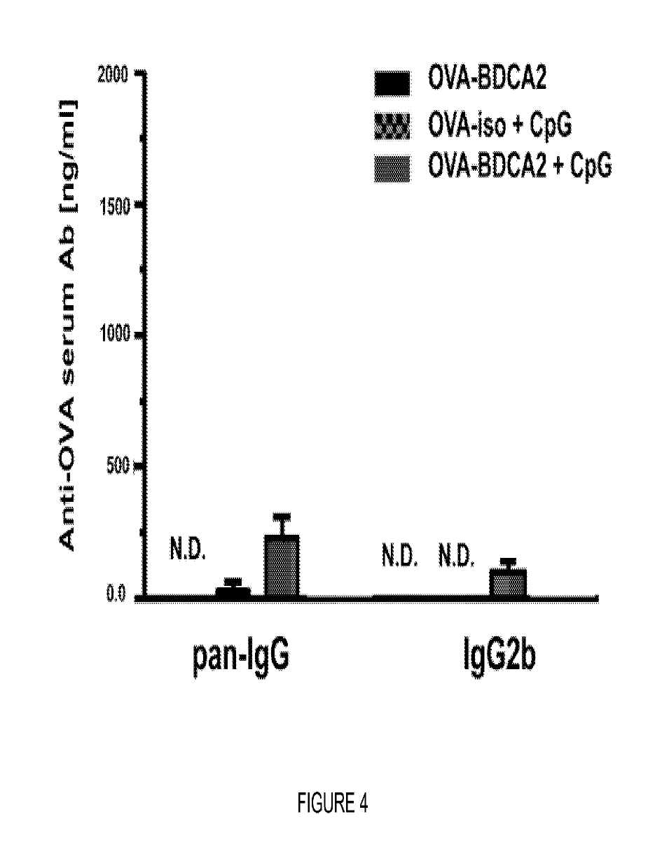

In one such study, OTII OVA-specific CD4+ T cells were inoculated into B6.BDCA2 Tg mice and one day later mice were inoculated i.v. with 10 μg OVA-BDCA2 mAb UW80.1 alone or in the presence of the adjuvant CpG-B (50 μg) or with 10 μg OVA conjugated to a mouse IgG1 non-binding isotype control mAb (G28-1 anti-human CD37, OVA-Iso) with CpG-B. IgG and IgG2b Ab responses 10 days after immunization were determined by ELISA. The data are shown in FIG. 4. Little or no specific IgG Ab was induced after targeting Ag to BDCA2, but the addition of an adjuvant did induce some but not high levels of specific Ab. N.D.=not detectable. Thus, without adjuvant, targeting Ag to BDCA2 does not induce Ab responses in vivo, but the addition of adjuvant can lead to Ag-specific Ab responses.

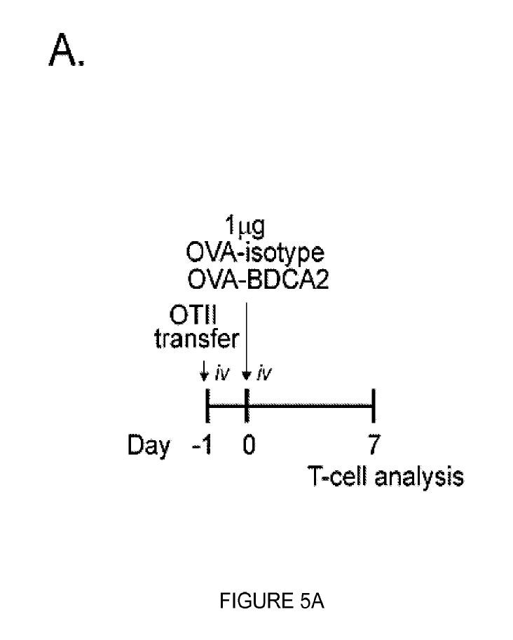

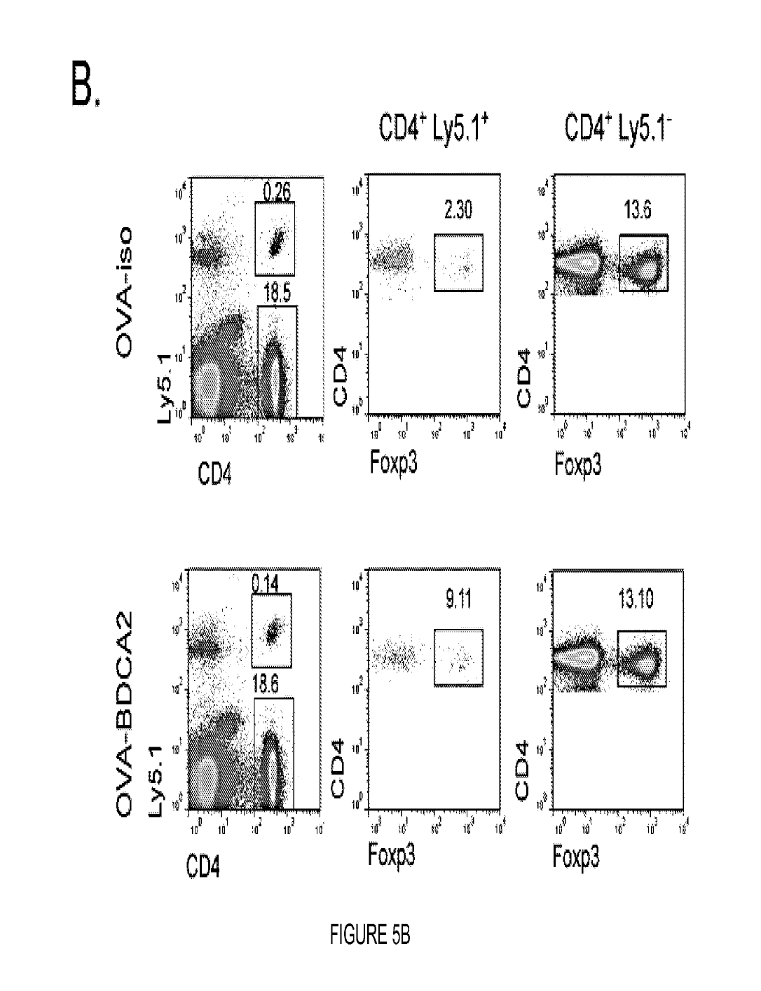

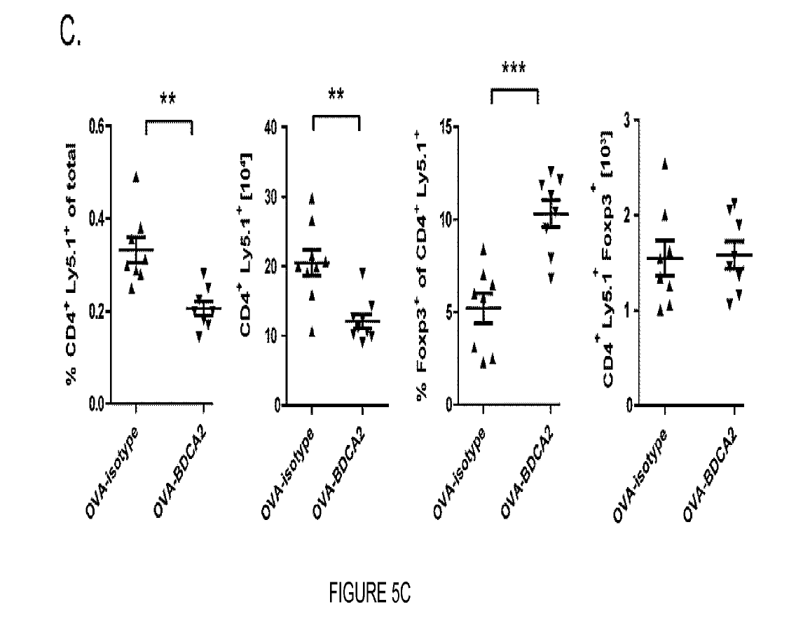

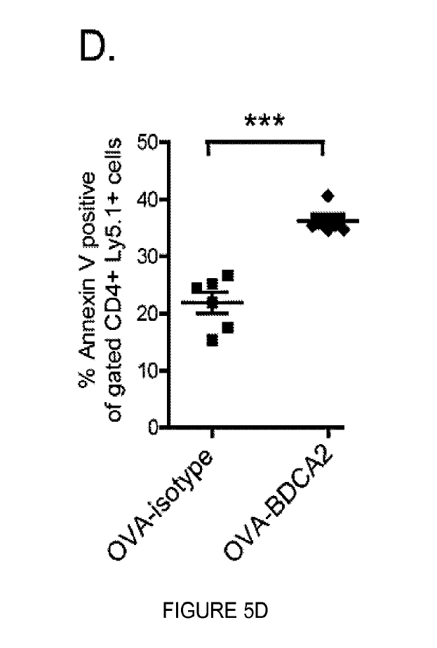

In a further experiment, mice were inoculated with OTII OVA-specific CD4 T cells on day −1 and 24 hrs later were inoculated i.v. with 1 μg of OVA-anti-BDCA2 or OVA-Iso (FIG. 5A) Flow cytometric analyses illustrated in FIG. 5B and summarized in FIG. 5C show that at day 7 the frequency and number of Ly5.1+ OTII cells were reduced in the OVA-anti-BDCA2 treated mice compared to OVA-Iso controls and that there was no decrease in the CD4+ Ly5.1-non-Tg T cells. Importantly, there was no decrease evident in the OT-II LY5.1+ cells expressing FoxP3 (FIGS. 5B and 5C), which led the proportion of CD45.1+ Foxp3+ cells to increase (FIG. 5B). Thus, the FoxP3+ regulatory CD4 T cells (Tregs) were not reduced but the Foxp3− CD4 T cells were. As a result, the proportion of Tregs in the Ag specific CD4 T cell population increases after Ag targeting to BDCA2 vs OVA-Iso (9.11% vs. 2.3%). Those proportions did not change in the Ly5.1-population. Thus, the effects of BDCA2-targeting are on CD4 T cell subsets are Ag-specific.

Injection of OVA-anti-BDCA2 compared to injection of OVA-Iso led to an increase in the percentage of dying Ag-specific CD4+ T cells. The data in FIG. 5D show that 7 days after Ag targeting via the method used above that there are more Annexin V+ cells (an indicator of cell death) in the population of OTII CD4+ Tg T cells from BDCA2 targeted mice vs. controls. ** p<0.01 and *** p<0.001. Similar results were obtained using alternative methods for detection of apoptosis, including staining with Mitotracker™ (data not shown). Thus, targeting to BDCA2 is a novel method for inducing deletion of Ag-specific CD4+ T cells, while at the same time retaining numbers of Ag-specific CD4+ Treg cells.

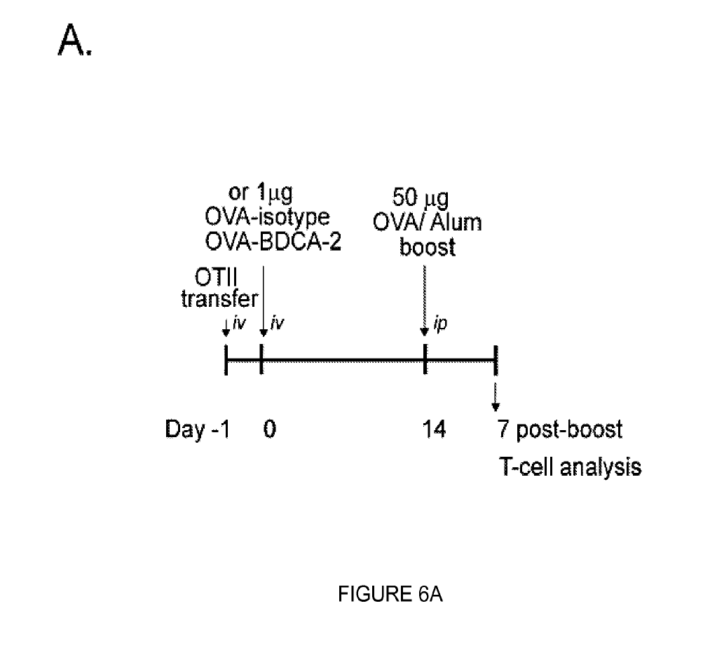

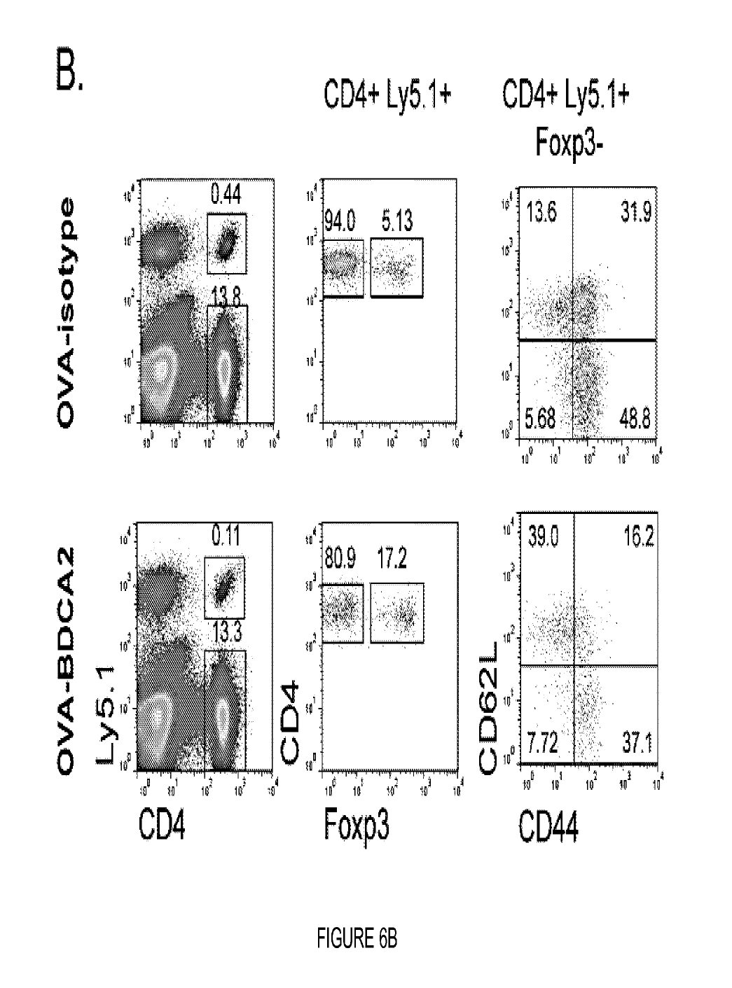

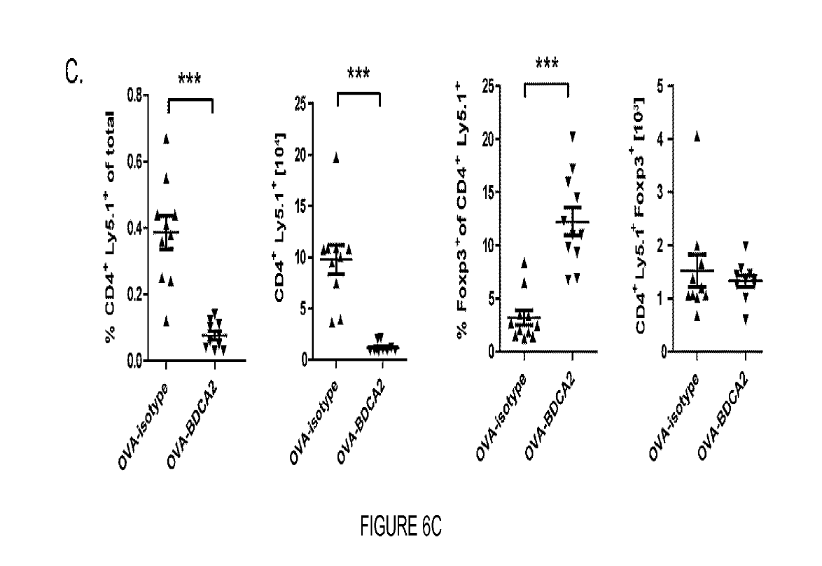

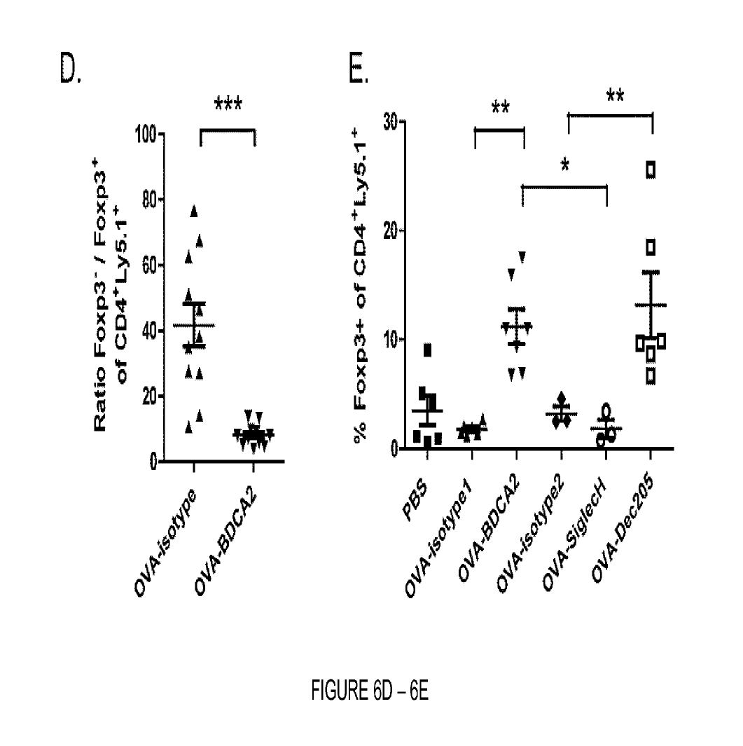

In another experiment mice were injected day −1 with OTII CD4+ T cells i.v. and on day 0 i.v. with 1 ug of OVA-isotype or OVA-BDCA2 mAb; 14 days later mice received a boost with a high dose intraperitoneally of OVA/precipitated in the Alum adjuvant, and 7 days later mice were analyzed for their frequencies and numbers of Ag-specific T cells (FIG. 6A). The mice primed with OVA-anti-BDCA2 compared to OVA-Iso immunized controls, after a secondary challenge had a dramatic decrease in the percentage and numbers of total Ly5.1+ CD4+ OTII cells (FIGS. 6B and 6C). However, once again the numbers of Foxp3+ CD4+ Ag-specific T cells did not decrease and as a result the overall frequency of Foxp3+ CD4+ Ag-specific T cells increased ((FIGS. 6B and 6C). The ratio of Foxp3-Ag-specific CD4+ T cells to Foxp3+ Ag-specific CD4+ T cells dropped significantly (FIG. 6D), demonstrating that a greater proportion of the Ag-specific CD4+ T cells were Foxp3+ T cells. These data show that after BDCA2 targeting even after a strong secondary challenge with Ag in adjuvant, the proportion of Foxp3+ Tregs cells is increased.

Surprisingly, the increased frequency of Foxp3+ CD4+ Ly5.1+ cells after targeting to BDCA2 was not evident when Ag was targeted to another receptor expressed on pDCs, mouse SiglecH (FIG. 6E). OVA was coupled to anti-Siglec H, anti-Dec205 (which binds to a receptor expressed on CD8+ myeloid DCs, or IgG2b isotype control mAb and injections and boosts were done as in FIG. 6A. After targeting to SiglecH and boosting with OVA/Alum, there was no change in the frequency of Foxp3+ CD4+ Ag-specific T cells, unlike after targeting to BDCA2 or targeting to Dec205 (FIG. 6E). Thus, the changes in Treg cell frequencies is not simply due to targeting to pDCs, but is unique to targeting to BDCA2 on pDCs.

The shift in the frequency of Foxp3+ CD4+ Ag-specific T cells after targeting OVA to BDCA2 and boosting with Ag in alum (FIG. 6A) was not dependent on the Alum adjuvant and the intraperitoneal route, but was present after boosting with OVA in a different adjuvant, complete Freund's adjuvant (CFA) and a subcutaneous inoculation (data not shown). Thus, after targeting Ag to BDCA2, the frequency of Foxp3+ CD4+ Ag-specific T cells is increased even after re-exposure of Ag in more than one adjuvant and more than one route.

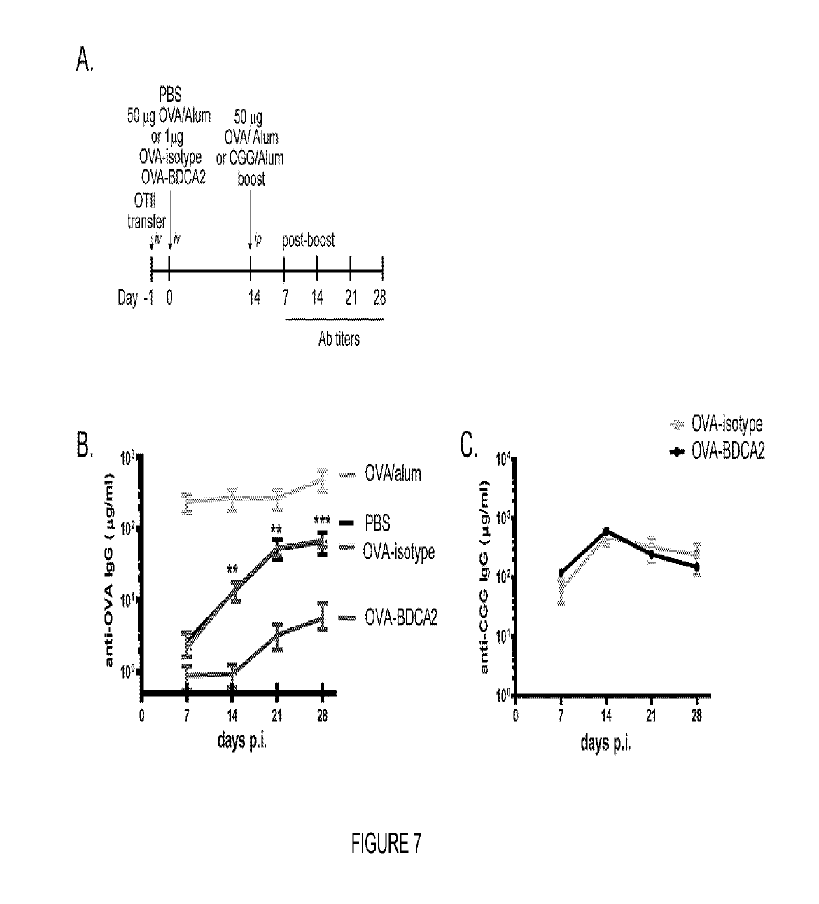

The fact that targeting to BDCA2 leads to a higher proportion of the Ag-specific CD4+ T cells that are Foxp3+ Tregs and that the Foxp3+ Tregs cell frequency is higher even after a secondary challenge with Ag suggested that targeting to BDCA2 may induce Ag-specific tolerance. To test this, groups of mice were injected day −1 with OTII CD4+ T cells i.v. and on day 0 intraperitoneally with 50 μg of OVA/precipitated in the Alum adjuvant (positive control) or i.v. with PBS (negative control), 1 ug of OVA-isotype mAb (negative control) or 1 ug of OVA-BDCA2 mAb; 14 days later mice were immunized with OVA/precipitated in the Alum adjuvant or with chicken gammaglobulin (CGG) precipitated in the Alum adjuvant and were bled 7, 14, 21 and 28 days thereafter (FIG. 7A).

Mice that had received only 1 microgram of OVA-anti-BDCA2, when boosted 14 days later with OVA/Alum adjuvant, had significantly reduced (**p<0.01) Ab responses to OVA compared to controls primed with OVA-Iso or PBS. Mice primed with OVA/Alum, as expected had strong responses to a secondary challenge with OVA/Alum (FIG. 7B). These data show a highly significant reduction after Ag re-challenge in the Ag-specific Ab responses in mice primed with OVA coupled to anti-BDCA2 compared to the isotype or PBS control group, strongly suggesting that Ag-specific tolerance is induced by targeting to BDCA2. To test if the reduction in Ab responses in BDCA2 targeted mice was in fact Ag-specific, a set of mice inoculated with OVA-anti-BDCA2 or OVA-Iso were boosted with CGG in Alum rather than with OVA in Alum (FIG. 7A, 7C). The mice re-challenged with a different Ag-CGG in Alum instead of OVA in Alum-had a normal Ab response to CGG, comparable to the isotype group (FIG. 7C). Thus, the significant reduction in Ab responses after targeting Ag to BDCA2 on pDCs is Ag-specific and a form of Ag-specific tolerance.

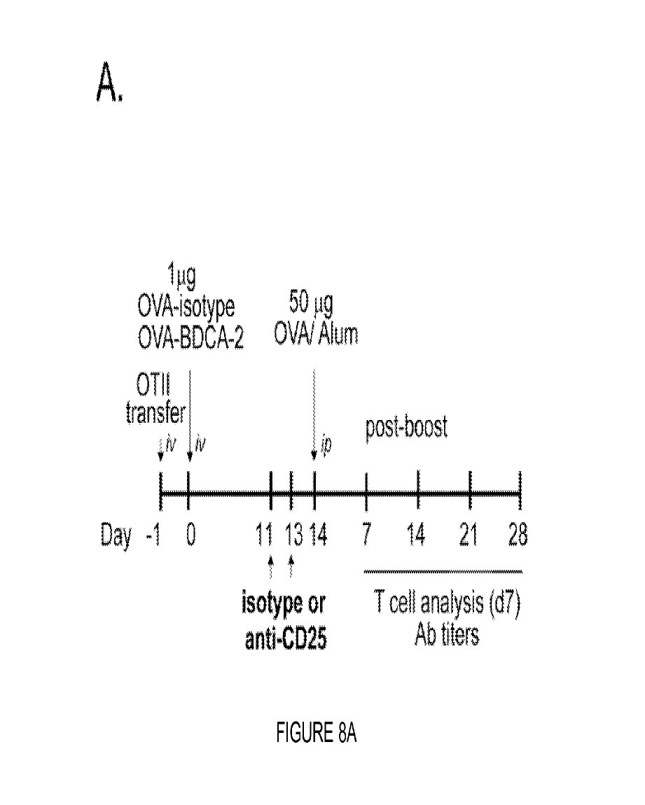

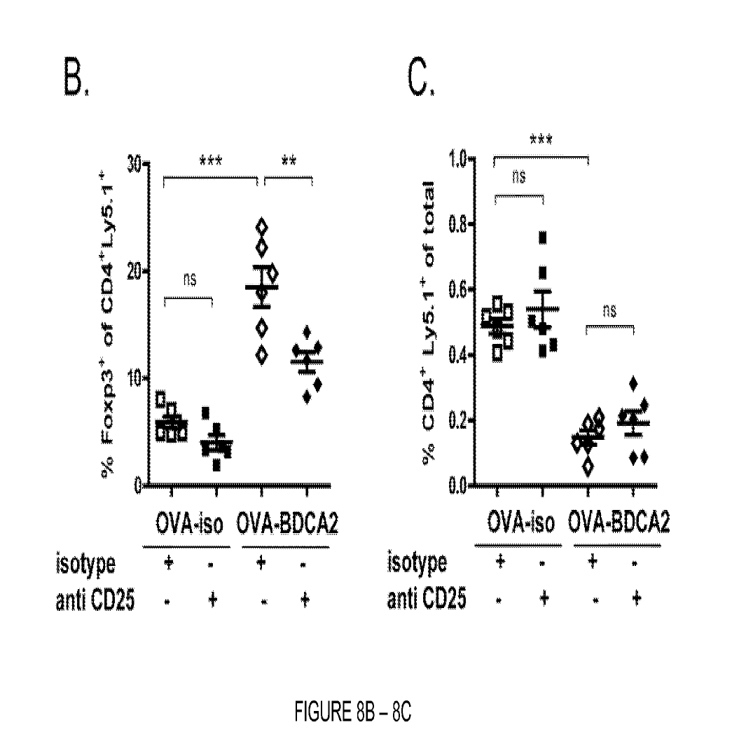

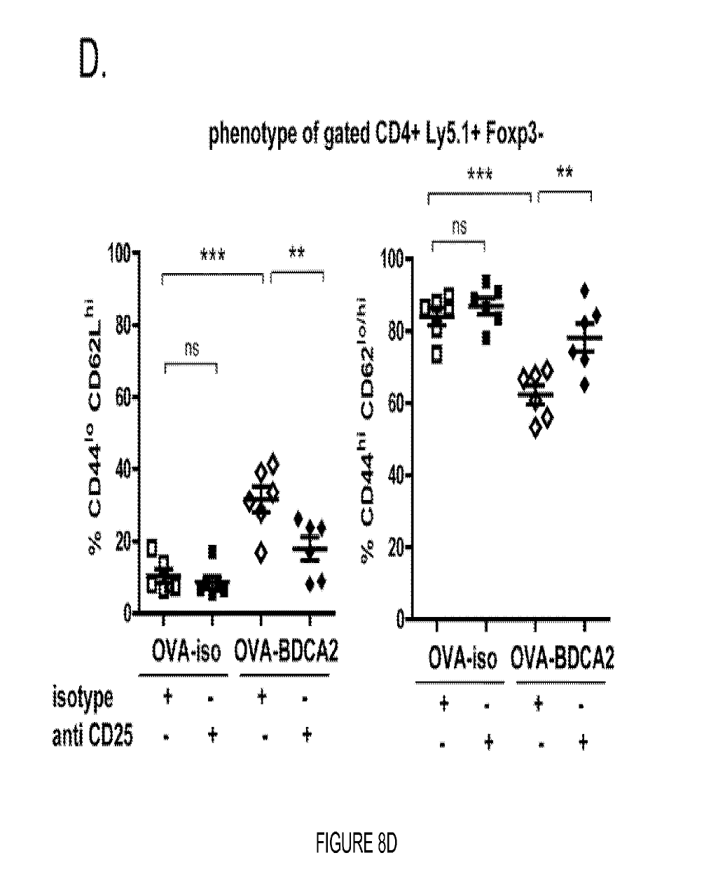

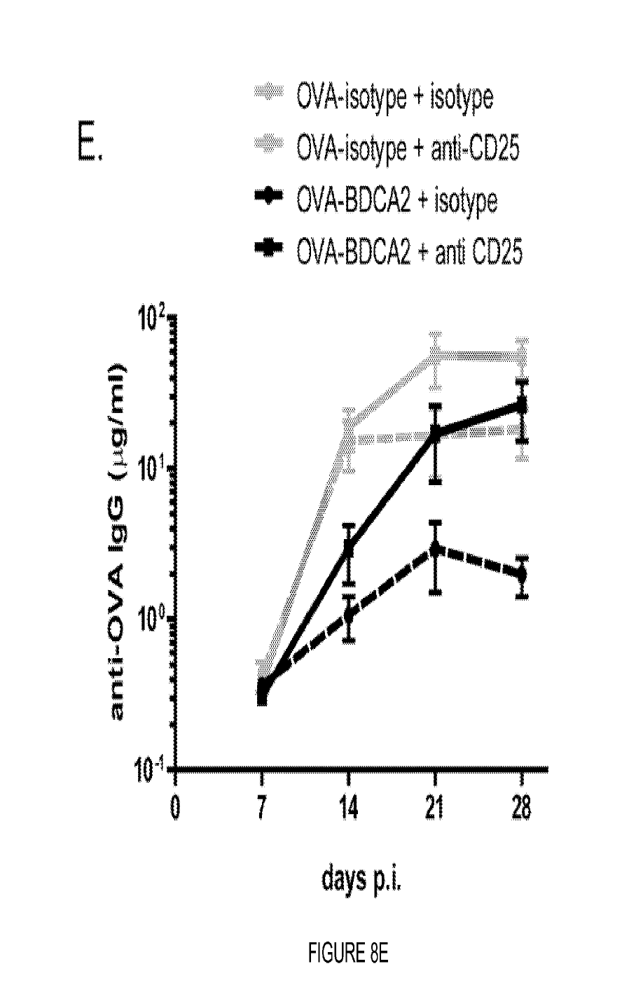

The facts that targeting to BDCA2 leads to a higher proportion of the Ag-specific CD4+ T cells that are Foxp3+ Tregs and that the Foxp3+ Tregs cell frequency is higher even after a secondary challenge with Ag suggested that targeting to BDCA2 may induce Ag-specific tolerance dependent on Ag-specific CD4+ Treg cells. An established method for depleting Treg cells in vivo is to inject mice with anti-CD25 mAb that binds to CD25+ Tregs and eliminates them. Therefore, after inducing Ag-specific tolerance via OVA-anti-BDCA2, we injected mice twice with a rat anti-CD25 mAb PC61.5.3 to reduce T regs or with a rat isotype control mAb (HRPN). These mice were then boosted with OVA/Alum and either followed for Ab responses or sacrificed 7 days later and assessed for CD4 T cell levels (FIG. 8A). Anti-CD25 Ab was injected i.p. 3 and 1 day prior to the boost. The inoculation of anti-CD25 led to a decrease in the frequency of Foxp3+ T regs (FIG. 8B) but did not alter the overall levels of Ag-specific CD4+ Ly5.1+ T cells (FIG. 8C). The inoculation of anti-CD25 led to a decrease in the frequency of Foxp3-CD44^{lo}CD62L^{hi} naïve T cells and an increase in the frequency of Foxp3-CD44^{hi}CD62L^{lo/hi} effector and memory T cells (FIG. 8D). These data demonstrate that the anti-CD25 treatment, as expected reduced levels of Foxp3+ T reg cells, and also increased the levels of effector CD4+ T cells. The depletion of Foxp3+ Ag-specific T cells led to restoration of the Ag-specific Ab responses (FIG. 8E). Unlike mice targeted with Ag to BDCA2 and treated with isotype control mAb-that had reduced Ag-specific Ab responses-mice targeted with Ag to BDCA2 and treated with anti-CD25 had normal Ag-specific Ab responses. Thus, the Ag-specific tolerance induced by targeting Ag to BDCA2 on pDCs requires CD25+ Treg cells.

To determine further the effect of adjuvant on the Ag-specific tolerance induced by targeting Ag to BDCA2 on pDCs, we carried out experiments outlined in FIG. 9A. Mice were injected day −1 with OTII CD4+ T cells i.v. and on day 0 i.v. with either PBS, 1 ug of OVA-isotype or OVA-BDCA2 mAb; alone or with 50 μg of the TLR-7 adjuvant R848. 14 days later mice received a boost intraperitoneally of 50 μg OVA/precipitated in Alum, and then 14 days later mice were analyzed for Ag-specific serum Ab levels (FIG. 9B). The mice inoculated with OVA-anti-BDCA2, as expected, had significantly lower Ab responses than mice inoculated with OVA-Iso. However, OVA-BDCA2 mice also inoculated with R848 made significantly more Ag-specific Ab. Thus, treatment with a TLR adjuvant at the time of antigen delivery breaks BDCA-2 induced Ag-specific immune tolerance and induces significant amounts of Ag-specific Ab.

We claim:

1. A composition, comprising:
(a) a blood dendritic cell antigen 2 (BDCA2) targeting molecule; and
(b) a heterologous antigen directly attached to the BDCA2 targeting molecule.

2. The composition of claim 1, wherein the composition includes no adjuvant.

3. The composition of claim 1, wherein the heterologous antigen is a polysaccharide or a glycolipid antigen.

4. The composition of claim 1, wherein the heterologous antigen is a polypeptide antigen.

5. The composition of claim 1, wherein the heterologous antigen is an antigen that can cause an undesirable immune response, an allergen or an autoimmune antigen.

6. (canceled)

7. The composition of claim 1, wherein the BDCA2 targeting molecule is a BDCA2 antibody or antibody fragment.

8. The composition of claim 7, wherein the BDCA2 antibody or antibody fragment comprises UW80.1 anti-BDCA2 antibody.

9. An isolated nucleic acid encoding the composition of claim 4.

10. A nucleic acid vector comprising the isolated nucleic acid of claim 9.

11. A recombinant host cell comprising the nucleic acid vector of claim 10.

12. An isolated UW80.1 anti-BDCA2 antibody, deposited with the American Type Culture Collection (ATCC) under Accession Number ______.

13. A hybridoma expressing UW80.1 anti-BDCA2, deposited with the American Type Culture Collection (ATCC) under Accession Number ______.

14. A pharmaceutical composition, comprising:
(a) the composition of claim 1; and
(b) a pharmaceutically acceptable carrier.

15. (canceled)

16. A method for treating or limiting development of a disorder, comprising administering to an individual at risk of a disorder or having a disorder, an amount effective to treat or limit development of the disorder of the composition of claim 1, or a pharmaceutically acceptable salt thereof.

17. (canceled)

18. The composition of claim 1, wherein the composition further comprises an adjuvant.

19. The composition of claim 18, wherein the heterologous antigen is a polysaccharide, polypeptide, or a glycolipid antigen.

20. (canceled)

21. The composition of claim 18, wherein the heterologous antigen is pathogen-specific antigen or a tumor antigen.

22. The composition of claim 18, wherein the BDCA2 targeting molecule is a BDCA2 antibody or antibody fragment.

23. The composition of claim 22, wherein the BDCA2 antibody or antibody fragment comprises UW80.1 anti-BDCA2 antibody.

24. A method for treating or limiting development of a disorder, comprising administering to an individual at risk of a disorder or having a disorder, an amount effective to treat or limit development of the disorder of the composition of claim 18, or a pharmaceutically acceptable salt thereof.
